(19) United States
(12) Patent Application Publication
(10) Pub. No.: US 2010/0115642 A1
(43) Pub. Date: May 6, 2010

(54) Xenogenic immune system in a non-human mammal

(75) Inventors: Kees Weijer, Amsterdam (NL); Nicolas Legrand, Amsterdam (NL)

Correspondence Address: EDWARDS ANGELL PALMER & DODGE LLP, P.O. BOX 55874, BOSTON, MA, 02205, US

(73) Assignee: Academisch Ziekenhius Bij de Universiteit Van Amsterdam

(21) Appl. No.: 12/455,633

(22) Filed: Jun. 3, 2009

Related U.S. Application Data
(63) Continuation of application No. PCT/NL2007/050623, filed on Dec. 5, 2007.

(30) Foreign Application Priority Data
Dec. 5, 2006 (EP) 06125413.2

(51) Int. Cl. A01K 67/027; C12N 15/01; C12N 5/00; C12Q 1/02

(52) U.S. Cl. 800/18; 800/21; 800/13; 435/325; 435/29

(57) ABSTRACT

The present invention relates to a method for providing a xenogenic immune system in an immunodeficient non-human mammal, to the obtained animal and to several uses of this animal, among other for producing xenogenic T cells.

### FIELD OF THE INVENTION

The present invention relates to a method for providing a xenogenic immune system in an immunodeficient non-human mammal, to the obtained animal and to several uses of this animal, among other for producing xenogenic T cells.

### BACKGROUND OF THE INVENTION

Reliable humanized immunodeficient animal models are required as preclinical animal models in order to test the impact on human immune system of new drugs, treatments, vaccines and other kinds of therapeutical interventions. Additionally, this kind of humanized animal model can be advantageously used for the production of human immune cells such as T cells for therapeutic purposes.

Several humanized animal models have been developed so far. All of them are suboptimal. For example, two groups have described sublethally irradiated new born BALB/c RAG2 (Recombination Activating Gene 2) and IL2Rγ (Interleukin2 Receptor gamma chain) deficient mice inoculated with hematopoietic progenitors (Traggiai E et al, 2004, Science, 304:104-107 and Gimeno R, et al, 2004, Blood, 104: 3886-3893). This recipient mice exhibit profound immunodeficiency and lack murine T, B, and NK cells. The use of newborn mice, instead of adult mice, leads, to considerable improvement of the engraftment by human progenitor cells and gives rise to multilineage reconstitution of the animals by human myeloid and lymphoid cells. Human T cells develop in situ in the mouse thymus, which can contain 2-10×10¹⁶ human thymocytes 4 to 8 weeks after stem cell inoculation. The developing human T cells repopulate the peripheral lymphoid organs of the mouse and all major human subpopulations are observed. However, this animal model is suboptimal at least for human T cell development and survival. A normal mouse thymus contains 100-200×10⁶ cells. Therefore, this animal model is limited as to the number of T cells present in the thymus: hypothetically it could contain between 10-100 more human T cells than what has been observed. In addition, in vivo stimulation of human T cells in this model can lead to peripheral T cell depletion.

Therefore, there is still a need for a method for providing an improved xenogenic immune system in a non-human mammal, that do not exhibit all the drawbacks of earlier animal models.

### DESCRIPTION OF THE INVENTION

### Method

In a first aspect, the invention relates to a method for providing a xenogenic immune system in a non-human mammal, said method comprising the following steps:
a. providing an immunodeficient non-human mammal as recipient, preferably an adult immunodeficient non-human mammal;
b. providing two xenogenic compositions, the first one comprising parts of xenogenic thymus and the second one comprising xenogenic hematopoietic progenitor cells as donor cells,
c. carrying out a total body sub-lethal irradiation in the non-human mammal of step a;
d. injecting clodronate-containing liposomes to the non-human mammal of step a;
e. engrafting the first xenogenic composition of step b comprising parts of xenogenic thymus in the non-human mammal of step a and,
f. as a last step introducing the second xenogenic composition of step b comprising xenogenic hematopoietic progenitor cells as donor cells in the non-human mammal of step a.

In the context of this invention, xenogenic immune system means an immune system from an organism or taxonomical species that has different taxonomical classification than the non-human recipient mammal. Normally the non-human recipient mammal would have shown an immunological reaction to the xenogenic compositions if it would not have been rendered immunodeficient.

### Immunodeficient Non-Human Mammal

An immunodeficient non-human mammal means a non-human mammal mutant, either man-made or naturally occurring, that has been rendered incapable of immune reaction. Typically, this non-human mammal lacks T, B, and NK cells and/or lacks functional T, B and NK cells and therefore does not mount an immunological response against the xenogenic compositions. Several immunodeficient non-human mammals have already been described. Non-limiting examples of immunodeficient non-human mammals that can be used in the method of the invention are the following. Non-limiting examples are given below of immunodeficient non-human mammals that lack T, B and NK cells:
BALB/c RAG2 and IL2Rγ deficient mice (Traggiai E et al, 2004, Science, 304:104-107 and Gimeno R, et al, 2004, Blood, 104: 3886-3893),
RAG2 and IL2Rγ deficient mice in a H-2^{d} mixed background (Weijer K et al, 2002, Blood, 99: 2752-2759 and Rozemuller H. et al, Exp. Hematol. 2004, 32:1118-1125),
NOD/SCID and IL2Rγ deficient mice (Ito M. et al, Blood, 2002, 100: 3175-3182; Hiramatsu et al, Blood, 2003, 102: 873-880),
RAG2 and IL2Rβ deficient mice (Suzuki et al, Science, 1995, 268: 14721476; Suzuki et al., J. Exp. Med., 1997, 185: 499-505) mice,
Nude mice (Flanagan S P et al, Genet. Res., (1966), 8:295-309, Kindred, B. et al, (1971) Eur. J. Immunol., 1:59-61, Pelleitier M et al, (1975), Methods Achiev. Exp. Pathol., 7:149-166) lack functional thymus, and can therefore be used in combination with any of the previous deficiencies (RAG2 and IL2Rγ or NOD/SCID or RAG2 and IL2Rβ) when production of humanized mice without human T cell development in the mouse thymus is wanted.

In addition, it is to be noted that each time a RAG2 deficient mouse is mentioned in this whole invention, this mouse may be RAG1 deficient as alternative to RAG2 or in combination with RAG2.

Alternatively, the immunodeficient non-human mammal may lack functional T, B and NK cells. Non-limiting examples are given below:
NOD/SCID mice (Non Obese Diabetis) (Melkus M. W., et al, 2006, Nature Medicine, 12:1316-1322, Rozemuller H. et al, Exp. Hematol. 2004, 32:1118-1125). These mice still show development of NK cells with partially impaired function. In order to get completely rid of the NK cell activity, the mice are treated with CD122/IL-2Rβ depleting antibody (Kerre et al., Blood, 2002, 99: 1620-1626) or anti-asialo GM1 antiserum (Yoshino et al., Bone Marrow Transplant., 2000, 26: 1211-1216).
NOD/SCID (Non Obese Diabetis/Severe Combined Immune Deficiency) and RAG2 deficient mice (Shultz L. D. et al, 2000, The Journal of Immunology, 164: 2496-2507). The same features apply to this strain.
Mice in the beige genetic background show impaired NK cell function and beige SCID mice are therefore susceptible to human hematopoietic stem cell engraftment (Kamel-Reid et al., Science, 1988, 242: 1706-1709);
   Immunodeficient non-human mammal may be prepared by conventional techniques for those skilled in the art or may be obtained by purchase or gift. Preferably, the immunodeficient non-human mammal is an adult. An adult non-human mammal is herein understood to mean a full-grown mammal that, preferably, is able to reproduce itself. Using an adult animal is preferred since the method of the invention might be too heavy for newborn animals. In addition, it is preferred that the growth of the animal is stabilized in order to ensure rapid and proper vascularisation of the engrafted first xenogenic composition containing parts of xenogenic thymus.

In a first preferred embodiment, the immunodeficient non-human mammal is deficient in at least the following genes:
RAG2 and IL-2Rγ (or IL-2Rβ) or
NOD/SCID and IL-2Rγ (or IL-2Rβ);
   More preferably, the immunodeficient non-human mammal is deficient in RAG2 and IL2Rγ (or IL-2Rβ). Even more, preferably, the immunodeficient non-human mammal is deficient in RAG2 and IL2Rγ. When using this animal in the method of the invention the best results were obtained as to the number of T cells and their longevity. Furthermore, RAG2 and IL2Rγ deficient mammals do not develop thymomas as do NOD/SCID mice, enabling long term in vivo studies. Even more preferably, the RAG2 and IL2Rγ deficient animal used is an adult.

The immunodeficient non-human mammal used in the method of the invention may further be deficient in other genes and/or may be transgenic for other genes. As an example, the immunodeficient non-human mammal may get enforced expression of human MHC molecules by transgenesis, as already done for instance with class I HLA-A2 (Pascolo S. et al., J. Exp. Med., 1997, 185: 2043-2051) or class II HLA-DR2 MHC molecules (Madsen L. S. et al., Nat. Genet., 1999, 23: 343-347). This is advantageous since it permits to select the T cell repertoire based on human determinants. As a preferred example of additional gene deficiency, the immunodeficient non-human mammal animal is further deficient for the flk2 gene (Mackarehtschian K. et al, (1995), Immunity, 3: 147-161). This is advantageous for carrying out the method of the invention, since the receptor tyrosine kinase flk2 is involved in myeloid differentiation, among other macrophage production. As another example of additional gene deficiency, the immunodeficient non-human mammal has further a CD11c-DTR. In these mice, CD11c⁺ phagocytes expressing the diphtheria toxin receptor (DTR) under the control of the CD11c promoter may be depleted from the animals by injection of the diphtheria toxin (S. Jung et al. In vivo depletion of CD11c(+) dendritic cells abrogates priming of CD8(+) T cells by exogenous cell-associated antigens. Immunity (2002), 17:211).

Therefore, when each of these types (flk2 deficient and/or CD11c-DTR background) of immunodeficient non-human mammal is used, it is to be expected that one of the pretreatment steps defined later as the injection of clodronate-containing liposome (step d) is not needed.

Alternatively or in combination with first preferred embodiment, the immunodeficient non-human mammal provided in step a is a mouse and the xenogenic compositions provided in step b originate from a human, rat, pig or non-human primate. The mouse is preferably a BALB/c (white) mouse. This type of mice is preferred since it seems to give better results in the method of the invention. The method of the invention may be carried out using a newborn mouse or an adult mouse. Preferably, the method is carried out on a mouse being between 3 and 24 weeks old, more preferably between 7 and 12 weeks old. In a more preferred embodiment, the method of the invention is carried out using an adult mouse. An adult mouse is at least 7 weeks old, more preferably at least 8 weeks old.

More preferably, the immunodeficient non-human mammal is an adult mouse and/or the xenogenic compositions originate from a human. Alternatively or in combination with first preferred embodiment, the immunodeficient non-human mammal provided in step a is a rat and the xenogenic compositions provided in step b originate from a human, mouse, pig or non-human primate. More preferably, the immunodeficient non-human mammal is an adult rat and/or the xenogenic compositions originate from a human.

Alternatively or in combination with first preferred embodiment, the immunodeficient non-human mammal provided in step a is a pig, more preferably an adult pig and the xenogenic compositions provided in step b originate from a human, mouse, rat or non-human primate. More preferably, the immunodeficient non-human mammal is an adult pig and/or the xenogenic compositions originate from a human.

### Xenogenic Composition Comprising Parts of Thymus (First Xenogenic Composition Defined in Step b)

Alternatively or in combination with previous preferred embodiments as earlier defined herein, the invention relates to a further preferred embodiment, wherein the first xenogenic composition comprises (parts of) xenogenic thymuses. More preferably, this xenogenic composition further comprises (parts of) xenogenic liver tissue. Liver tissue may be replaced by bone marrow. Some hematopoietic progenitors (characterized as CD34⁺ if the xenogenic individual is a human) may already be present in liver tissue and/or bone marrow and/or thymus. They are not counted as part of the hematopoietic progenitors to be introduced. Part of thymus and liver tissue and bone marrow used can be either of fetal or post-natal origin or both. Preferably, parts of thymus and liver tissues are recognized via their own characteristic colour, location and/or texture. Liver has typically a pastel red colour. The thymus is attached to the thoracic cage and has a characteristic lobular structure. For isolation of bone marrow, whole bones are isolated directly.

Each mouse typically receives approximatively 1 to 4 pieces of thymus and optionally 1 to 4 pieces of liver. Each piece is approximatively a cube of 1 to 2 mm side. Preferably, the bone marrow is rendered accessible trough longitudinally cut bone.

If the availability of fetal thymus and liver tissues is limited, alternatively the origin of both tissues may be post-natal. Post-natal thymus can for example be isolated during a cardiac surgery intervention. Preferably, post-natal means two years of age or less.

In step b, in a most preferred embodiment, both xenogenic compositions preferably originate from the same species. More preferably, both xenogenic compositions originate from the same individual of this species. Even more preferably, both xenogenic compositions are both of human origin. Even more preferably, both compositions originate from the same human being.

Alternatively or in combination with earlier defined preferred embodiments, the xenogenic composition comprising parts of thymus further comprises parts of xenogenic spleen and/or parts of xenogenic skin. Xenogenic spleen and skin preferably originate from the same individual as the thymus, liver and bone marrow. Each piece of spleen is approximately a cube of 1 to 2 mm side, the surface of transplanted skin is approximately a square of around 5 mm side (after removal of an equivalent surface of recipient animal skin). Xenogenic spleen is advantageous for further improving the reconstitution of xenogenic B cells. Xenogenic skin is attractive to be present in the animal model prepared carrying out the method of the invention for specific applications of the animal model such as monitoring cutaneous immunization of the animal model for a specific administered infectious agent.

Alternatively or in combination with earlier defined preferred embodiments, the xenogenic composition comprising parts of thymus further comprises parts of other (fetal) organs. Any other (fetal) organ may be used as long as it provides a source of epithelial cells. For instance lung and/or gut may be used: a part of lung preferably used is 5×5×5 mm s.c., a part of gut preferably used is 1 cm long, longitudinally open. The presence of epithelial cells is important if one envisage to screen for drug or vaccine candidates when the pathogen requires the presence of epithelial cells for its own replication. An example of such a pathogen is HCMV.

### Xenogenic Composition Comprising Xenogenic Hematopoietic Progenitor Cells (Second Xenogenic Composition Defined in Step b)

Depending on the species of the organism the hematopoietic progenitor cells originate, the skilled person knows which marker(s) may be used to isolate these cells from the organism. The origin of the xenogenic hematopoietic progenitor cells present in the second xenogenic composition of step b is not crucial for performing the method of the invention. What is crucial is that these cells express a specific marker, which characterizes hematopoietic progenitor cells of a given species. In a preferred embodiment, both xenogenic compositions originate from a human being, more preferably from the same human being. In this preferred embodiment, the xenogenic human hematopoietic progenitor cells are characterized by the expression of the CD34 marker as commonly known by the skilled person. The origin of the xenogenic CD34⁺ human hematopoietic progenitor cells present in the second xenogenic composition of step b is not crucial for performing the method of the invention. What is crucial is that these cells express the marker CD34. Preferably, these cells do not express the marker CD38. As an alternative for the marker CD34, the marker CD133 may also be used to isolate and enrich for hematopoietic progenitors (Kobari L., et al, (2001), J. Hematotherapy and Stem Cell Res. 10: 273-281).

Alternatively or in combination with previous preferred embodiments earlier defined herein, the invention relates to a further preferred embodiment, wherein the xenogenic CD34⁺ hematopoietic progenitor cells present in the second xenogenic composition provided in step b are isolated from at least one of the following sources selected from the group consisting of: fetal liver, umbilical cord blood, bone marrow, hematopoietic stem cells differentiated from embryonic stem cells and mobilized peripheral blood. More preferably, these cells are isolated from fetal liver. The skilled person knows how to isolate and/or obtain such cells. Usually a Ficoll step is carried out following by an enrichment step for CD34⁺ using a commercial kit to this end and/or fluorescence associated cell sorting (Becton Dickinson, USA, or Milteniy Biotech, CD34⁺ separation kit, Germany). For example, xenogenic fetal liver-derived progenitor cells can be obtained as described in Gimeno et al (Gimeno R, et al, 2004, Blood, 104: 3886-3893). As another example, xenogenic umbilical cord blood progenitors can be obtained as described in Traggiai et al (Traggiai E et al, 2004, Science, 304: 104-107).

### Pretreatment

In order to optimize the efficiency of the final step f. of introduction of the second xenogenic composition as provided in step b. comprising xenogenic hematopoietic progenitor cells as donor cells, the immunodeficient recipient animal of step a needs to be pretreated. The pretreatment comprises several steps (c, d, and e) which may be carried out in any possible chronological order:
c. carrying out a total body sub-lethal irradiation in the non-human mammal of step a;
d. injecting clodronate-containing liposomes to the non-human mammal of step a;
e. engrafting the first xenogenic composition of step b comprising parts of xenogenic thymus in the non-human mammal of step a.
   The chronological order of the pretreatment steps may be: c, d, e or c, e, d or d, c, e or d, e, c, or e, c, d or e, d, c. Preferably, the chronological order is d, c, e or c, d, e, meaning the engraftment of the first xenogenic composition is preferably carried out after the total body sub-lethal irradiation and the injection of clodronate-containing liposomes.

One of these pretreatment steps is a total body sub-lethal irradiation (step c). Irradiation is a common procedure before hematopoietic transplantation. This treatment creates space in the stem cell niche by depleting radio-sensitive murine bone marrow cells. Typically, the irradiation received is ranged between 2 and 4 Gray, or between 2 and 3 Gray. Preferably, the irradiation received is about 3.0 Gray, or about 2.8 Gray. The source of irradiation used is not critical.

Another of these pretreatment steps is an injection of clodronate-containing liposomes (step d). This treatment is carried out to deplete phagocytes from the immunodeficient recipient animal. This treatment was for the first time described by Van Rooijen N. et al (Van Rooijen N et al, (1989), Journal of Leukocyte Biology, 45:97-104 and Van Rooijen N et al, (1994), Journal of Immunological Methods, 174:83-93). Several studies on immunodeficient animals used such treatment (Van Rijn R. S., et al, 2003, Blood, 102: 2522-2531, Legrand N. et al, 2006, J. Immunol., 176: 2053-2058 or Rozemuller H et al, 2004, Exp. Hematol., 32: 1118-1125). Preferably, this treatment is carried out by intra peritoneal or intra venous injection of 100 to 200 μl of a liposomal preparation containing 2.5 mg/ml clodronate. Usually one single treatment is necessary. A treated animal comprises substantially no phagocytes as defined later herein. If the animal used is deficient for flk2 and/or have a CD11c-DTR background as mentioned before, this step of the pretreatment may be avoided.

Another of these pretreatment steps is the engraftment of the first xenogenic composition comprising parts of xenogenic thymus (step e). The engraftment may be carried out under the kidney capsule of the mouse. Alternatively, the engraftment may be intra muscular, intra peritoneal or subcutaneous. In a preferred embodiment, the engraftment is subcutaneous. Subcutaneous engraftment has already been successfully used in the SCID mice (Mc Cune J. M., et al, 1988, Science, 241: 1632-1639). It is a relatively easy engraftment technique which has the advantage of being less invasive for the engrafted recipient animal than other classical non-subcutaneous engraftment techniques. Briefly, an incision is made on the skin on the back of the mouse, parts of xenogenic thymus and optionally liver or others tissues as earlier defined herein are inserted under the skin with forceps. Some Matrigel™ Matrix (basement membrane matrix, Becton Dickinson) could be applied to glue all pieces together in the same area under the skin and to improve vascularisation. Finally the skin is closed again.

As a last step, the second xenogenic composition comprising xenogenic hematopoietic progenitor cells as donor cells provided in step b. is introduced into the non-human mammal immunodeficient animal of step a. The last step is therefore preferably carried out on the pretreated animal. Pretreated means the pretreatment as defined under the paragraph entitled “pretreatment” has been carried out on the animal. Preferably, the second xenogenic composition is introduced intra-venously into the animals. The skilled person will understand that it is possible to introduce the second xenogenic composition into the animals by other routes, e.g. lymphatics, lymphoid organs (spleen, liver). Preferably, the introduction of the second xenogenic composition is carried out between one and 15 days after the pretreatment (c, d, e) as described above. More preferably, between one and 10 days, even more preferably between two and 5 days. Hematopoietic progenitor cells present in the composition to be introduced have been earlier defined. Preferably, if the hematopoietic progenitor are CD34⁺ cells, at least 10⁵ CD34⁺ cells are introduced per mouse. More preferably, at least 10⁶ CD34⁺ cells are introduced per mouse. In another preferred embodiment, at least 10⁴ CD34⁺CD38⁻ cells are introduced per mouse. More preferably, at least 10⁵ CD34⁺CD38⁻ cells are introduced per mouse. This second xenogenic composition comprising the CD34⁺ cells may further comprise a suitable medium. The suitable medium may be RPMI (GibcoBRL).

### Animal

In a second aspect, the invention provides a non-human mammal obtainable by the method of the invention. Preferred animals are as herein defined above. A preferred animal includes a non-human mammal deficient in RAG2 and IL2Rγ or in RAG2 and IL2Rβ, and/or being a mouse, and/ being an adult mouse, and/or comprising substantially no phagocytes and/or comprising xenogenic immune T and/or B cells. A non-human mammal is preferably obtainable by the method of the invention and is deficient in RAG2 and IL2Rγ or in RAG2 and IL2Rβ and is engrafted with a first xenogenic composition comprising parts of xenogenic thymuses and parts of liver tissue and with a second xenogenic composition comprising xenogenic hematopoietic progenitor cells.

As used herein the term “comprising substantially no phagocytes” preferably means that as a result of the clodronate-containing liposomes treatment as earlier defined herein or as the result of the use of a non-human mammal animal wherein this treatment may be avoided (flk2 deficient and/or CD11c DTR background), the amount of phagocytes has decreased dramatically during several weeks (transient dramatic decrease) and/or is preferably not detectable during at least approximately one day till approximately one week and/or is preferably not detectable at all (definitive elimination). In case, a non-human mammal used is flk2 deficient and/or has a CD11c DTR background, the amount of phagocytes is preferably not detectable at all (definitive elimination). The number of phagocytes is preferably determined by cell count and/or flow cytometry analysis in the lymphoid organs of the immunodeficient non-human mammal used. Examples of murine markers specific for phagocytes that could be used for flow cytometry analysis include MAC-1 or F4/80.

As used herein the term “comprising xenogenic immune T and/or B cells” preferably means that a non-human mammal animal as defined herein comprises a number of xenogenic immune T and/or B cells which is as close as possible to a wild type non-human mammal. For example, if the non-human mammal used is a mouse: the thymus may have about 100 millions thymocytes, bone marrow 20 millions cells per femur, 100 millions splenocytes composed by 60% B cells and 30% T cells. Preferably the immunodeficient mouse of the invention has:
approximately between 5-10 million xenogenic, preferably human cells per femur (mostly B cells); and/or
approximately between 1-5 million xenogenic thymocytes, preferably human thymocytes in the mouse thymus, and multiples of 50 million xenogenic preferably human thymocytes in the thymic implant (xenogenic composition 1); and/or
approximately between 1-5 several million xenogenic, preferably human splenocytes composed by approximately 70-90% B cells and 1-20% T cells.
   The number of xenogenic immune T and B cells is preferably assessed by cell count and/or flow cytometry analysis in the lymphoid organs of the immunodeficient non-human mammal used. In case, the xenogenic immune T and B cells are human cells, example of specific human T cells markers that could be used in flow cytometry analysis includes CD3. For human B cells, an example of a specific human marker is CD 19.

A most preferred animal obtainable by the method of the invention is a RAG2 and IL2Rγ deficient mouse subcutaneously engrafted with parts of human thymuses and liver tissues and containing human CD34⁺ progenitors. This animal model for the production of a xenogenic immune system constitutes an improvement over known animal models since it allows both a quantitative and qualitative improvement of the recovered xenogenic immune cells. The main effect is as expected an accumulation of T cells (20-30 fold increase in absolute cell numbers) with a longer survival capacity, as described more precisely in the supporting data enclosed in this document. The presence of the thymic transplant in hematopoietic stem cells inoculated Rag-2 IL-2Rγ_{c} deficient mice [HIS (Rag/γ)] does not only impact positively on human T cell life-span and accumulation. The absolute B cell numbers in the spleen of HIS (Rag/γ) mice with a thymic transplant were also increased around 2-fold, as compared to a 25-fold increase in T cell numbers (see Table in the results section).

This accumulation of B cells is expected, due to several factors in relation to T cell accumulation. First, T cells produce soluble factors which participate to B cell proliferation and function, and vice-versa, with stimulatory, differentiation and chemo-attraction effects. As far as B cell differentiation is concerned, it is known that the so-called “helper” CD4⁺ T cells are required for immunoglobulin isotype switch. An increased amount of long-lived T cells ultimately leads to higher chances to produce and accumulate switched B cells, in particular “memory” IgG-producing B lymphocytes. Second, it is known that secondary lymphoid organs (e.g. spleen) are disorganized at least partially during lymphopenic conditions, which are observed in the classical HIS (Rag/γ) mice without thymic transplant. The accumulation of lymphocytes is leading to increased structural organization of the lymphoid organs, and this contributes to increased survival of lymphocytes in situ. Last, the human thymic transplant itself contributes to the global “welfare” of human lymphocytes in the mouse environment. Indeed, it is known that thymic epithelial cells produce growth and survival factors involved in development and survival of lymphocytes, of which IL-7 is a major contributor. Therefore, IL-7 production by epithelial cells of the thymic transplant and subsequent release in the circulation may impact positively on the global survival state of human lymphocytes developing in humanized mice.

It can be excluded that other human cell lineages are also positively affected, since IL-7 is also a stem cell survival factor. Furthermore, a new subset of thymus-derived Natural Killer (NK) cells expressing the IL-7 receptor was recently identified (Vosshenrich C. et al, 2006, Nat. Immunol., 7: 1217-1224). The additional thymic transplant will therefore contribute to increased production and seeding of IL-7R⁺ human NK cells in HIS (Rag/γ) mice.

Concomitant accumulation of both arms of adaptative immunity has direct consequences on immunological functionality of human cells in such animals, due to improved structural organization, B-T cell cross-talk and survival of the human lymphocytes. As a consequence, improved B and T cell immune responses are logically expected in HIS (Rag/γ) mice harbouring a thymic transplant.

### Methods Wherein the Animal is Used

In a third aspect, the invention provides a method of producing xenogenic immune cells using the non-human mammal preferably obtainable by the first method of the invention as defined in the second aspect of the invention, and, optionally recovering the xenogenic immune cells. Preferably, in this method the most preferred animal as defined herein above is used. The xenogenic immune cells are preferably T and/or, B and/or NK cells.

After transplantation, the recipient animal is maintained in specific pathogen free (SPF) conditions. At least 6 weeks post transplantation (preferably 7-8 weeks), a valuable amount of xenogenic immune cells, including T and B cells, can be recovered.

These T cells are functional since they can be isolated and stimulated ex vivo. Furthermore, they can also mount (at least partially) immune responses against pathogens. The functionality of these T cells may be assayed as it has already been described in Legrand N. et al (Legrand N. et al, (2006), J. Immunol., 176: 2053-2058).

These B cells are functional since they can be isolated and may at least partially switch to form in particular “memory” IgG-producing B lymphocytes. The switch of these B cells may be assayed by classical techniques as ELISA (Enzyme Linked Immuno Sorbent Assay) for IgM and IgG. Partly means at least 5% of the B cells are able to produce IgG, preferably at least 10%, more preferably at least 15%, even more preferably at least 30%, even more preferably at least 40%, even more preferably at least 50%, and most preferably more than 50%.

Accordingly, the invention relates to a method of producing T and/or B cells using the non-human mammal preferably obtainable by the first method of the invention and as defined in the second aspect of the invention, and, optionally recovering the xenogenic immune cells, wherein:
the T cells are functional and can be stimulated ex vivo, and/or
the B cells are functional and can at least partly switch to IgG-producing lymphocytes.

In a fourth aspect of the invention, there is provided a method of screening a compound for its effect on xenogenic immune cells, preferably T and/or B cells wherein the non-human mammal preferably obtainable by the first method of the invention and as defined in the second aspect of the invention is exposed to a control compound and the effect of the compound on the xenogenic immune cells, preferably T cells is analyzed. Optionally, the non-human mammal has been infected by a given infectious agent.

As an example, the compound is a cytokine or a putative cytokine, a drug, a vaccine, a (monoclonal) antibody. Its effect on xenogenic immune cells can be assayed in the animal model of the invention. Preferably, the immune cells are T and/or B cells.

Alternatively or in combination with a method of screening a compound as defined in the fourth aspect, there is provided in a fifth aspect of the invention a method for testing the effect of a potential treatment, on xenogenic immune cells, preferably on T and/or B cells.

Optionally, in all aspects mentioned in this section “methods wherein the animal is used”, the non-human mammal has been infected by an infectious agent like HIV (Human Immunodeficiency Virus), HCMV (Human CytoMegalo Virus) or hepatitis viruses including HCV (Hepatitis C Virus). Alternatively, autoimmune diseases such as Rheumatoid arthritis or colitis ulcerosa may be induced in this animal model. Alternatively, leukemia, lymphoma or other human tumors (e.g. melanoma) can be induced in the non-human mammal of the invention. Subsequently, the efficacy of compounds and/or treatments can be tested on the xenogenic immune cells, preferably T cells. Preferably, the effect on T cells is analyzed as described in Legrand N et al (2006). Preferably, in this method the most preferred animal as defined in the previous section entitled animal is used.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

### DESCRIPTION OF THE FIGURES

FIG. 1: Flow cytometry analysis of HIS (BALB-Rag/γ) thymus 8 weeks post-reconstitution. Human (huCD45⁺) thymocytes are stained for the expression of the CD4 and CD8 co-receptors. The percentage of each thymocyte subpopulation is indicated in the quadrants.

FIG. 2: Presence of a T cell population with phenotypic characteristics of regulatory T (T_{reg}) cells. Cytometry analysis shows the expression of the IL-2Rα/CD25 molecules on blood (left panel) and spleen (right panel) CD4⁺ T cells in HIS (BALB-Rag/γ) mice. Glucocorticoid-induced tumor necrosis factor receptor family-related gene (GITR/TNFRSF18) expression is also shown for spleen CD4⁺CD25⁺ T cells.

FIG. 3: T cell status in HIS (BALB-Rag/γ-T/L) versus mice (FTi HIS). The left panel shows the frequency of T cells in the spleen of control HIS mice vs. FTi HIS mice. The right panel shows BrdU incorporation in T cells of each group after a 24-hour pulse. Horizontal bars indicate the mean value (n=4, **p<0.01).

### EXAMPLE

### Results

### The “Human Immune System” (HIS) (BALB-Rag/γ) Mouse Model

The “Human Immune System” (HIS) mouse model has been recently described by two groups that inoculated hematopoietic progenitors into sublethally irradiated newborn BALB/c Rag-2^{-/-}γ_{c}^{-/-} mice (1, 2). These recipient mice exhibit profound immunodeficiency, and lack murine T, B and NK cells. The use of newborn mice, instead of adult animals, leads to considerable improvement of the engraftment by human progenitor cells, and gives rise to multilineage reconstitution of the animals by human myeloid and lymphoid cells (3). Since the recipient mice are Balb/c (white) Rag-2^{-/-}γ_{c}^{-/-} mice, in opposition to C57Bl/6 Rag-2^{-/-}γ_{c}^{-/-} mice which do not get efficiently reconstituted, the model is referred to as HIS (BALB-Rag/γ) mice (3), but is also known as “human adaptative immune system Rag-2^{-/-}γ_{c}^{-/-} mice” (huAIS-RG) (4).

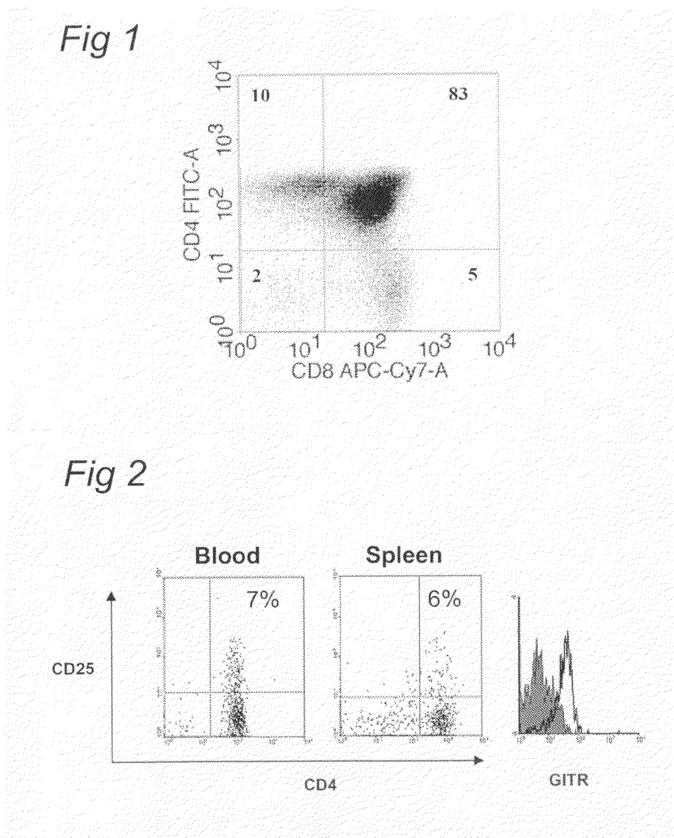

Human T cells develop in situ in the mouse thymus, which can contain 2-10·10⁶ human thymocytes 4-8 weeks after stem cell inoculation (FIG. 1). The developing T cells repopulate the peripheral lymphoid organs of HIS (BALB-Rag/γ) mice, and all major subpopulations, including regulatory T cells (T_{reg}) (FIG. 2), are observed. Despite the fact that HIS (BALB-Rag/γ) mice represent a clearly improved system, as compared to the previously described models, it is important to note that it is still suboptimal. This is especially the case for human T cell development (normal mouse thymus contains 100-200×10⁶ cells) and survival. We have for instance shown that T cell stimulation in vivo can lead to peripheral T cell depletion in HIS (BALB-Rag/γ) mice, and that high human T cell turn-over takes place in non-manipulated animals (5). This may be due to lack of proper T cell survival-inducing factors, e.g. human MHC molecules, cytokines (IL-2, IL-7, IL-15), growth factors (3).

### A New Optimized Model: HIS (BALB-Rag/γ-T/L) Mice

### Methods:

Three different approaches have been combined to obtained an optimized humanized mouse system: (a) the previously described HIS (BALB-Rag/γ) mouse model (3); (b) the ability of clodronate-containing liposomes to induce deletion of mouse phagocyting cells (e.g. macrophages) (6); and (c) the classical SCID-Thy/Liv model which is mainly based on human thymus transplantation into mouse hosts (7).

The HIS (BALB-Rag/γ-T/L) mice are made as follows:
adult BALB/c Rag-2^{-/-}γ_{c}^{-/-} mice receive one intra-venous injection of clodronate-containing liposomes (to deplete murine macrophages). This is also possible with adult H-2^{d} Rag-2^{-/-}γ_{c}^{-/-} mice in a mixed genetic background (8);
the animals are also subjected to sub-lethal irradiation (to create space in the stem cell niche and deplete murine bone marrow derived radio-sensitive cells);
one day after clodronate injection, a mixture of fetal liver and fetal thymus samples (from the same donor) is placed sub-cutaneously in the anesthetized animals;
the remaining fetal liver from the same donor is processed, the CD34⁺CD38⁻ hematopoietic stem cells enriched fraction is isolated and inoculated to the animals;

### Results:

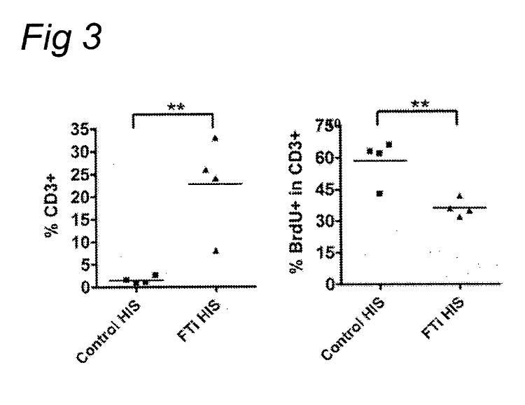

The sub-cutaneous thymic implant in the HIS (BALB-Rag/γ-T/L) mice is usually palpable by hand after few weeks. At 8 weeks post-transplantation, HIS (BALB-Rag/γ-T/L) mice contain more T cells in peripheral lymphoid organs, as compared to control HIS (BALB-Rag/γ) mice (FIG. 3—left), which can be seen as a consequence of increased T cell production in the transplant. Furthermore, these T cells exhibit a longer survival capacity, as assessed by BrdU incorporation (FIG. 3—right). This can be either a direct consequence of increased T cell production, increased synthesis of T cell survival factors (e.g. huIL-7 produced by the thymic transplant itself), or a combination of the two. Overall, the absolute number of T cells is largely increased in the spleen (×25), and other subsets (B cells) also beneficiate of the global increase in human cell engraftment (Table 1).

TABLE 1
Absolute human cell numbers in thymus and spleen of HIS
(BALB-Rag/γ-T/L) mice (×10⁶ cells).

| | THY all | THY | SPL all | SPL B | |
|---|---|---|---|---|---|
| | human | implant | human | cells | SPL T cells |
| Ctrl HIS | 1.3 ± 0.2 | N/A | 2.5 ± 1.5 | 1.5 ± 1.1 | 0.06 ± 0.04 |
| FTi HIS | 1.1 ± 0.2 | 102 ± 131 | 5.6 ± 2.5 | 2.9 ± 1.2 | 1.5 ± 0.9 |
| | | | (x2.2) | (x1.9) | (x25) |

Values (mean ± SD, n = 4) are given for control HIS (BALB-Rag/γ) mice (ctrl HIS) and HIS (BALB-Rag/γ-T/L) mice (FTi HIS).
THY: thymus;
SPL: spleen;
N/A: non applicable.

### Prospects:

The HIS (BALB-Rag/γ-T/L) mice exhibit a more stable T cell subset, and a B/T cell ratio closer to normal physiological conditions. The human engraftment is overall improved. It is therefore expected to be more even more suitable than HIS (BALB-Rag/γ) model for studies on human lymphocyte development, lymphocyte homeostasis and immunization.

### REFERENCES

1. Traggiai, E., Chicha, L., Mazzucchelli, L., Bronz, L., Piffaretti, J. C., Lanzavecchia, A., and Manz, M. G. Development of a human adaptive immune system in cord blood cell-transplanted mice (2004) Science 304, 104-7.
2. Gimeno, R., Weijer, K., Voordouw, A., Uittenbogaart, C. H., Legrand, N., Alves, N. L., Wijnands, E., Blom, B., and Spits, H. Monitoring the effect of gene silencing by RNA interference in human CD34+ cells injected into newborn RAG2−/− gammac−/− mice: functional inactivation of p53 in developing T cells (2004) Blood 104, 3886-93.
3. Legrand, N., Weijer, K., and Spits, H. Experimental models to study development and function of the human immune system in vivo (2006) J Immunol 176, 2053-8.
4. Chicha, L., Tussiwand, R., Traggiai, E., Mazzucchelli, L., Bronz, L., Piffaretti, J. C., Lanzavecchia, A., and Manz, M. G. Human Adaptive Immune System Rag2−/−{gamma}c−/− Mice (2005) Ann NY Acad Sci 1044, 236-43.
5. Legrand, N., Cupedo, T., van Lent, A. U., Ebeli, M. J., Weijer, K., Hanke, T., and Spits, H. Transient accumulation of human mature thymocytes and regulatory T cells with CD28 superagonist in “human immune system” Rag2(−/−) gammac(−/−) mice (2006) Blood 108, 238-45.
6. van Rijn, R. S., Simonetti, E. R., Hagenbeek, A., Hogenes, M. C., de Weger, R. A., Canninga-van Dijk, M. R., Weijer, K., Spits, H., Storm, G., van Bloois, L., Rijkers, G., Martens, A. C., and Ebeling, S. B. A new xenograft model for graft-versus-host disease by intravenous transfer of human peripheral blood mononuclear cells in RAG2−/− gammas−/− double-mutant mice (2003) Blood 102, 2522-31.
7. McCune, J., Kaneshima, H., Krowka, J., Namikawa, R., Outzen, H., Peault, B., Rabin, L., Shih, C. C., Yee, E., Lieberman, M., and et al. The SCID-hu mouse: a small animal model for HIV infection and pathogenesis (1991) Annu Rev Immunol 9, 399-429.
8. Weijer, K., Uittenbogaart, C. H., Voordouw, A., Couwenberg, F., Seppen, J., Blom, B., Vyth-Dreese, F. A. and Spits H. Intrathymic and extrathymic development of human plasmacytoid dendritic cell precursors in vivo. (2002) Blood 99, 2752-2759.

1. Method for providing a xenogenic immune systems in a non-human mammal, said method comprising the following steps:
a. providing an immunodeficient non-human mammal as recipient, preferably an adult immunodeficient non-human mammal,
b. providing two xenogenic compositions, the first one comprising parts of xenogenic thymus and the second one comprising xenogenic hematopoietic progenitor cells as donor cells,
c. carrying out a total body sub-lethal irradiation in the non-human mammal of step a,
d. injecting clodronate-containing liposomes to the non-human mammal of step a,
e. engrafting the first xenogenic composition of step b comprising parts of xenogenic thymus in the non-human mammal of step a,
f. as a last step introducing the second xenogenic composition comprising xenogenic hematopoietic progenitor cells as donor cells of step b in the non-human mammal of step a.

2. The method according to claim 1, wherein the immunodeficient non-human mammal is deficient in at least the following genes:
RAG2 and IL2Rγ, or
RAG2 and IL2Rβ or
NOD/SCID and IL 2rγ

3. The method according to claim 1, wherein the immunoefficient non-human mammal provided in step of a claim 1 is a mouse, more preferably an adult mouse and the xenogenic compositions provided in step b of claim 1 originate from a human, rat, pig or non0human primate, preferably from a human.

4. The method according to claim 1, wherein the immunodeficient non-human mammal provided in step a of claim 1 is a rate, more preferably an adult rat and the xenogenic compositions provided in step b of claim 1 originate from human, mouse, pit or non-human primate, preferably from a human.

5. The method according to claim 1, wherein the immunodeficient non-human mammal provided in step a of claim 1 is a pig, more preferably an adult pig and the xenogenic compositions provided in step b of claim 1 originate from a human, mouse, rat or non-human primate, preferably from the human.

6. The method according to claim 1, wherein the xenogenic donor cells present in the second xenogenic compositions provided in step b of claim 1 are human CD34⁺ hematopoietic progenitor cells.

7. The method according to claim 6, wherein the CD34⁺ hematopoietic progenitor cells are isolated from at least one of the following sources selected from the group consisting of: fetal liver, umbilical cord blood, bone marrow and mobilized peripheral blood.

8. The method according to claim 1, wherein the first xenogenic composition provided in step b of claim 1 comprises parts of xenogenic thymuses and part of liver tissue.

9. A non-human mammal obtainable by the method of any one claims 1.

10. A non-human mammal preferably according to claim 9, deficient in RAG2 and IL2Rγ or in RAG2 and IL2Rβ engrafted with a first xenogenic composition comprising parts of xenogenic thymuses and parts of liver tissue and with a second xenogenic composition comprising xenogenic hematopoietic progenitor cells.

11. A non-human mammal according to claim 9, wherein the non-human mammal is a mouse.

12. A non-human mammal according to claim 11, wherein the mouse is an adult mouse.

13. A non-human mammal according to claim 9, wherein the non-human mammal comprises substantially no phagocytes.

14. A non-human mammal according to claim 9, wherein the non-human mammal comprises xenogenic immune T and/or B cells.

15. A method of producing xenogenic immune cells using the non-human mammal as defined in claim 9, optionally recovering the xenogenic immune cells.

16. The method according to claim 15, wherein the xenogenic immune cells are T and/or B cells.

17. The method according to claim 16, wherein:
the T cells are functional and can be stimulated ex vivo, and/or
the B cells are functional and can at least partly switch to IgG-producing lymphocytes

18. A method of screening a compound for its effect on xenogenic immune cells, wherein the non-human mammal as defined in claim 9 is exposed to a control compound and the effect of the compound on the xenogenic immune cells, preferably T cells is analyzed.

19. The method of claim 18, wherein the compound is a drug or vaccine, and optionally the non-human mammal as defined in claim 9 has been infected by an infectious agent.
